(19) United States
(12) Patent Application Publication
(10) Pub. No.: US 2025/0065101 A1
(43) Pub. Date: Feb. 27, 2025

(54) A SYSTEM TO TREAT HEART FAILURE WITH PRESERVED EJECTION FRACTION (HFpEF)

(71) Applicant: Royal College of Surgeons in Ireland, Dublin (IE)

(72) Inventors: Aamir Hameed, Dublin (IE); Andrew Malone, Dublin (IE); Grainne Malone, Dublin (IE)

(21) Appl. No.: 18/723,137

(22) PCT Filed: Dec. 21, 2022
(86) PCT No.: PCT/EP2022/087380

§ 371 Date: Jun. 21, 2024

(30) Foreign Application Priority Data
Dec. 22, 2021 (EP) 21217170.6

(51) Int. Cl. A61M 60/17; A61M 60/237; A61M 60/416; A61M 60/531; A61M 60/806; A61M 60/861; A61M 60/865; A61M 60/873; A61M 60/88

(52) U.S. Cl. CPC A61M 60/17; A61M 60/237; A61M 60/416; A61M 60/531; A61M 60/806; A61M 60/861; A61M 60/865; A61M 60/873; A61M 60/88; A61M 2230/04

(57) ABSTRACT

A system to treat heart failure with preserved ejection fraction (HFpEF) is described. The system comprises a blood pumping device configured for implantation in a left ventricle of a heart of a subject, an anchoring assembly for anchoring the blood pumping device to a wall of the left ventricle, and a controller configured to modify the output parameters of the blood pumping device so as to activate and deactivate the blood pumping device in a pattern synergistic with a cardiac cycle of the subject comprising activation during ventricular diastole and deactivation during ventricular systole.

### FIELD OF THE INVENTION

The present invention relates to a system to treat heart failure with preserved ejection fraction (HFpEF). The invention also relates to a method of treating heart failure with preserved ejection fraction (HFpEF), and a method of relieving or preventing secondary pulmonary hypertension.

### BACKGROUND TO THE INVENTION

Heart failure is defined as inability of the heart to supply adequate blood to the body. As the demographics suggest, with increasing ageing population, prevalence of heart failure is also increasing. The cardinal manifestations of HF are dyspnea and fatigue, which may limit exercise tolerance, and fluid retention, which may lead to pulmonary and/or splanchnic congestion and/or peripheral oedema.

Heart cycle has two phases; a contraction phase when heart pumps the blood to the whole body and the relaxation phase when heart is filled with blood. Heart failure associated with the contraction phase (systole) includes HF with reduced ejection fraction (HFrEF), a condition commonly caused by ischemic heart disease that leads to decrease in stroke volume and cardiac output which results in the activation of neurohormonal response in order to restore the normal cardiac output. Left ventricular assist devices have been developed to assist the heart during the contraction phase, including pump devices designed to assist the pumping of blood into the aorta during diastole (contraction), and contractile devices designed to be implanted between two walls of the left ventricle that reciprocate in a pattern synergistic with the heart rhythm to assist the contraction of the left ventricle during systole. Pump devices for treatment of HFrEF are described in US2016/000983 and U.S. Pat. No. 7,942,804.

Heart disease associated with the relaxation phase of the heart cycle (diastole) includes Heart failure with preserved ejection fraction (HFpEF). HFpEF is a clinical syndrome in which patients have symptoms and signs of HF with normal or near normal left ventricular ejection fraction (LVEF>50 percent). During early diastole phase of the cardiac cycle, the healthy LV acts as a ‘vacuum cleaner’ that enhances the suction particularly during exercise. In HFpEF, cardiomyocyte stiffness results in the loss of this relaxation enhancement, hence normal LV filling is dependent on high left atrial (LA) pressure to push blood into the LV. This pressure elevation can further cause atrial remodelling and secondary pulmonary hypertension, predisposing patients to develop atrial fibrillation and right ventricular (RV) dysfunction. Each 10 mm Hg increment in pulmonary artery pressure in patients with HFpEF was found to be associated with a 28% increase in 3-year mortality 3. Hence, there is a need to effectively control the death rate from HFpEF.

To date, no pharmacological treatment has yet been shown to reduce morbidity and mortality in patients with HFpEF in randomised clinical trials, mainly due to the pathophysiological heterogeneity of the disease. Current treatment strategies as per the American Heart Association (AHA) and European Society of Cardiology (ESC) focus on treating the comorbidities and use of diuretics to relieve congestion.

Transcatheter left to right interatrial shunt devices (REDUCE LAP-HF-I) have been proposed as a possible treatment for HFpEF, although right ventricular (RV) volume overloading and subsequent RV failure and worsening of secondary pulmonary hypertension may be an issue with these shunt devices.

A left ventricular implantable device that applies direct internal expansion forces to increase the LV volume (CORolla) has also been proposed as a possible treatment for HFpEF. In theory, expansive elements in the CORolla device could be used for the treatment of all forms of HFpEF, however performance of such a device would likely be heavily dependent on the degree of cardiac remodelling, which varies significantly over HFpEF phenotypes.

WO2020/081481 describes an implantable device to treat HFpEF comprising a pump that is attached to the mitral valve and is controlled to pump blood from the left atrium to the left ventricle during the cardiac cycle where the pump operates continuously between a low pumping speed (first current) during myocardial perfusion during diastole and a high pumping speed (second current) during the rest of the cardiac cycle. The constant operation of the pump is required as it is attached to the mitral valve and therefore prevents the mitral valve ever fully closing. Thus, during ventricular systole, when the mitral valve is normally fully closed, it is necessary to actuate the pump at high speed to prevent backflow of blood from the left ventricle into the left atrium. The low pumping speed during diastole is required to prevent compression of the left ventricle during diastole. Attaching the pump via a sewing cuff to the mitral valve is a complicated procedure. In addition, the device has a wide bore meaning that it would not be delivered percutaneously and would require heart surgery.

It is an objective of the invention to overcome at least one of the above-referenced problems.

### SUMMARY OF THE INVENTION

The objective is met by the provision of a system and method for treatment of HFpEF that employs a blood pumping device implanted in the left ventricle configured for sequential activation during diastole (to assist the left ventricle pull blood into the left ventricle from the right atrium through the open mitral valve) and deactivation during systole when the left ventricle is emptying. Thus, unlike the system of WO2020/081481 which operates continuously including during systole (to prevent backflow of blood), the system of the invention activates the blood pumping device only during ventricular diastole. This is made possible to implanting the blood pumping device in the left ventricle. A controller is operatively coupled to the blood pumping device and configured to activate and deactivate the blood pumping device in a pattern synergistic with the cardiac cycle of the subject comprising activation during ventricular diastole and deactivation during ventricular systole. The controller may receive signals from a sensor, which may form part of the blood pumping device or may be a separate sensor such as an ECG or atrial sensor. The blood pumping device generally comprises an impeller and ideally is an axial flow pump. In use, the blood pumping device is anchored to a wall of the left ventricle and positioned such that upon activation the pressure in the top of the left ventricle adjacent the mitral valve is reduced sufficiently to assist the drawing of blood into the left ventricle from the right atrium.

In a first aspect, the invention provides a system comprising:
a blood pumping device configured for implantation in a left ventricle of a heart of a subject;
an anchoring assembly for anchoring the blood pumping device to a wall of the left ventricle; and
a controller configured to modify the output parameters of the blood pumping device so as to activate and deactivate the blood pumping device in a pattern synergistic with a cardiac cycle of the subject comprising activation during ventricular diastole and deactivation during ventricular systole.

In any embodiment, the system is to treat heart failure with preserved ejection fraction (HFpEF).

In any embodiment, the system is to treat, relieve or prevent secondary pulmonary hypertension.

In any embodiment, the system is to treat a condition associated with impaired filling of the left ventricle during ventricular diastole.

In any embodiment, the system comprises at least one sensor in communication with the controller for detecting one or more parameters associated with the heart, wherein the controller is configured to modify the output parameters of the blood pumping device based on the one or more detected parameters received from the sensor.

In any embodiment, the controller is configured to modify the pump flow rate and/or the frequency of actuation of the blood pumping device.

In any embodiment, the controller is configured to modify the amplitude of the voltage of the power supply to the blood pumping device. When the pump comprises a rotor, varying the amplitude of the voltage modifies the speed of rotation of the rotor.

In any embodiment, the controller is configured to modify the frequency of the voltage duty cycle of the power supply to the blood pumping device. Varying the frequency of the voltage controls the activation and deactivation of the pump.

In any embodiment, the at least one sensor is selected from the group consisting of: a pressure sensor, a wireless pressure sensor, a mems pressure sensor, an artery pressure sensor, a cardiac output (CO) sensor, a blood pressure sensor, an ejection fraction of the left ventricle, a heart rate sensor, a motion sensor, an accelerometer, an ECG (Electrocardiogram) sensor, an O2 saturation sensor, a micro accelerometer, and a sonomicrometer. The pressure sensor can be a ventricular and/or atrial pressure sensor.

In any embodiment, the sensor is a heart pacing sensor.

In any embodiment, the sensor is an atrial or ventricular pressure sensor.

In any embodiment, the system comprises a heart pacing sensor and an atrial or ventricular pressure sensor.

In any embodiment, the sensor is integral with the blood pumping device.

In any embodiment, the sensor is coupled to heart tissue.

In any embodiment, the blood pumping device comprises an impeller.

In any embodiment, the blood pumping device comprises an axial flow pump.

In any embodiment, the blood pumping device comprises:
a housing comprising a fluid inlet, a fluid outlet, and a lumen extending through the housing from the fluid inlet to the fluid outlet;
a rotor disposed in the housing;
an impeller disposed on the rotor; and
a motor operably coupled to the rotor for rotation thereof upon activation,
   in which the blood pumping device is configured to draw fluid through the housing from the inlet to the outlet upon activation.

In any embodiment, the fluid inlet of the housing extends into the left atrium through the mitral valve. In this embodiment, the blood pumping device is configured to be completely contained within the left ventricle with only part of the fluid inlet extending into the left atrium.

In any embodiment, the blood pumping device comprises a fluidic extension conduit configured to provide fluidic communication from inside the left atrium to the fluid inlet of the housing.

In any embodiment, the fluidic extension conduit is detachably coupled to the fluid inlet of the housing.

In any embodiment, the fluidic extension conduit is flexible.

In any embodiment, the impeller is disposed on the rotor at a fluid outlet side of the housing.

In any embodiment, the housing is a tubular housing.

In any embodiment, the tubular housing comprises a first cylindrical part connected to a second cylindrical part by a plurality of struts.

In any embodiment, the inlet and outlet are aligned along a common axis.

In any embodiment, the blood pumping device comprises:
a first cylindrical part comprising the motor;
a second cylindrical part comprising the impeller;
a central part connecting the first cylindrical part and second cylindrical part comprising the fluid inlet; and
a fluid outlet disposed in the first cylindrical part or second cylindrical part,
   wherein the rotor extends from the motor to the impeller through the central part.

In any embodiment, the fluid inlet comprises one or more apertures in the central part of the housing.

In any embodiment, the second part comprises the fluid outlet. In this embodiment, rotation of the impeller pulls blood through the fluid inlet and through the second part and out of the fluid outlet.

In any embodiment, the second part of the housing has a diameter D1 that is greater than a diameter D2 of the first part of the housing. In any embodiment, a ratio of D1 to D2 is in a range of 3:2 to 5:2.

In any embodiment, the central part of the hosing has a frustoconical shape.

In any embodiment, the central part of the housing comprises a plurality of struts connecting the first and second parts of the housing.

In any embodiment, the fluid outlet is disposed in the first part. In this embodiment, the first part has a lumen for fluid to allow the fluid to pass the motor and exit through the fluid outlet.

In any embodiment, the motor is an electromagnetic motor comprising a stator disposed within the housing surrounding at least part of the rotor.

In any embodiment, the impeller comprises an axial hub and at least two vanes mounted to the hub, in which each vane has an elongated swept profile.

In any embodiment, each vane has a hub to tip ratio (v) of 0.20 to 0.30.

In any embodiment, the impeller comprises two vanes disposed on opposed sides of the hub.

In any embodiment, each vane has an axial length of 5 to 20 to mm, preferably 10 to 15 mm, and ideally about 12 to about 13 mm.

In any embodiment, each vane has a radial width of 1 to 10 mm, 3 to 7 mm, 4 to 6 mm, or ideally about 5 mm.

In any embodiment, each vane extends around the hub by a sweep angle of 70° to 140°, 80° to 120°, 90° to 120°, or about 100° to about 120°. The sweep angle refers to the angle that the vane sweeps around the hub. It can be seen in the end view shown in FIG. 6B where the vane can be seen to extends around the hub by about 100°.

In any embodiment, a clearance between the radial tip of each vane and the hub housing is 0.1 mm to 1.5 mm, 0.3 mm to 0.7 mm, 0.4 mm to 0.6 mm, and ideally about 0.5 mm.

In any embodiment, the controller is configured to receive heart rate data from a sensor and modify the output parameters of the blood pumping device in real time based on the received heart rate data.

In any embodiment, the controller is configured to compare the heart rate data with reference impeller rotational speed data, calculate an impeller rotational speed based on the comparison, and actuate the blood pumping device during diastole phases to rotate the impeller at the calculated impeller rotational speed.

In any embodiment, the blood pumping device is dimensioned for percutaneous delivery to the left ventricle of the heart inside a delivery catheter of up to 32 Fr, typically along a guidewire.

In any embodiment, the blood pumping device is configured for mounting on a guidewire.

In any embodiment, the anchoring assembly comprises one or more anchor elements (e.g., arms) coupled to the blood pumping device.

In any embodiment, the anchoring assembly is configured for mounting to a left ventricular septum, an apex of the left ventricle, or the left ventricular septum and the apex of the left ventricle.

In any embodiment, the anchoring assembly comprises a plurality of anchoring arms configured for adjustment from a stowed position suitable for percutaneous delivery to a deployed position in which the plurality of anchoring arms oppose the ventricular walls. In any embodiment, the anchoring arms are configured to be splayed radially outwardly upon deployment.

In any embodiment, each anchoring arm comprises a distal tissue engaging anchor such as, for example, a barb.

In any embodiment, the anchoring arms are attached to a distal end of the housing.

In any embodiment, the anchoring arms are configured for self-deployment. For example, each arm may be biased into a deployed position, and self-deploy when the device is advanced beyond a distal end of a delivery sheath.

In any embodiment, the anchoring assembly is configured to couple to the blood pumping device in-vivo.

In any embodiment, the anchoring assembly comprises an anchoring hub configured for coupling to the blood pumping device and a plurality of anchoring arms extending from the hub.

In any embodiment, the system further comprises electronic circuitry for setting the output parameters of the blood pumping device, wherein the electronic circuitry is coupled between the controller and the blood pumping device, and wherein the controller is configured to send control signals to the electronic circuitry to modify the output parameters of the blood pumping device.

In any embodiment, the controller is configured for implantation.

In any embodiment, the controller is configured for implantation under the skin of a subject's chest, typically sub dermally.

In any embodiment, the system further comprises a power unit associated with the blood pumping device.

In any embodiment, the power unit is associated with the electronic circuitry.

In any embodiment, the power unit is configured for implantation.

In any embodiment, the power unit is configured for implantation under the skin of a subject's chest, typically sub dermally.

In any embodiment, the controller and power unit (and optionally the electronic circuitry) are contained within a single implantable housing.

In any embodiment, the system comprises a power lead to provide electrical communication between the power unit and the blood pumping device, typically through the controller and electronic circuitry.

In any embodiment, the blood pumping device comprises a chamber and the power lead disposed (e.g., fully disposed) in the chamber in a spooled or wound configuration. In this embodiment, the blood pumping device is delivered to the left ventricle with the power lead spooled in the chamber. Once the blood pumping device is deployed, a proximal end of the power lead is retrieved using a medical device and delivered to the implanted power unit (typically via the controller) which may be implanted under the skin of a subject's chest.

In any embodiment, the system comprises an access sheath having a lumen configured for percutaneous delivery of the blood pumping device to the left ventricle.

In any embodiment, the system comprises a delivery shaft for the blood pumping device to advance the blood pumping device through the lumen of the access sheath.

In any embodiment, the delivery shaft and blood pumping device are configured for detachable coupling together. This allows the blood pumping device to be attached to the delivery shaft, advancement of the shaft through the access sheath to deliver the device, and then detachment of the delivery shaft from the blood pumping device.

In any embodiment, the system comprises a handle attached to a proximal end of the access sheath, typically via a first haemostasis valve.

In any embodiment, a proximal end of the delivery shaft is attached to the handle, typically via a second haemostasis valve.

In any embodiment, the handle comprises a first actuator to adjust the axial position of the delivery shaft relative to the access sheath.

In any embodiment, the handle comprises a second actuator to rotate the delivery shaft relative to the access sheath.

In any embodiment, the controller is configured for implantation within the left ventricle of the heart. In any embodiment, the controller is integral with, or operatively connected to, the blood pumping device.

In another aspect, the invention provides a method, for example a method of treating or preventing heart failure, for example treating a subject with a heart condition such as HFpEF, comprising the steps of:
delivering a blood pumping device inside a subject's heart and positioning the blood pumping device to draw blood from the left atrium into the left ventricle; and
activating and deactivating the blood pumping device in a pattern synergistic with the cardiac cycle of the subject comprising activation during ventricular diastole and deactivation during ventricular systole.

In any embodiment, the method comprises anchoring the blood pumping device to a wall of the left ventricle of the subject's heart.

In any embodiment, the method is a method of treating heart failure with preserved ejection fraction (HFpEF).

In any embodiment, the method is a method of relieving or preventing secondary pulmonary hypertension.

In any embodiment, the method comprises:
sensing with a sensor a heart rate of the subject; and
activating and deactivating the blood pumping device in a pattern (e.g., at a frequency) synchronous with the sensed heart rate of the subject.

In any embodiment, the method comprises modulating the frequency of activation and deactivation of the blood pumping device according to changes in the sensed heart rate over time.

In any embodiment, the step of modulating the frequency of activation and deactivation of the blood pumping device comprises modulating the frequency of the voltage duty cycle to the blood pumping device.

In any embodiment, the method comprises:
sensing with a sensor a blood pressure parameter the subject's heart, typically a blood pressure parameter of a left side of the heart, preferably a left atrial or left ventricular blood pressure parameter; and
during activation of the blood pumping device, activating the blood pumping device (e.g., modulating the speed of rotation of an impeller of the blood pumping device) at a pumping speed appropriate to the sensed blood pressure parameter.

In any embodiment, the method comprises modulating the pumping speed of the blood pumping device according to changes in the sensed blood pressure parameter over time.

In any embodiment, the step of modulating the pumping speed of the blood pumping device comprises modulating the amplitude of the voltage supply to the blood pumping device.

In any embodiment, the method comprises activating the impeller of the blood pumping device at an impeller rotational speed of 5,000 rpm to 50,000 rpm.

In any embodiment, the method comprises activating the impeller of the blood pumping device at an impeller rotational speed of 10,000 rpm to 40,000 rpm.

In any embodiment, the method comprises reducing the pressure in the left ventricle by at least 5-10 mmHg during ventricular diastole.

In any embodiment, the method comprises delivering the blood pumping device to the left ventricle percutaneously.

In any embodiment, the blood pumping device is percutaneously delivered to the left ventricle via a femoral vein-->iliac vein-->inferior vena cava--->right atrium--->interatrial septum--->left atrium--->left ventricle

In any embodiment, the method comprises implanting a sensor in communication with, or into, the heart of the subject. Typically, the sensor is a blood pressure parameter sensor. In one embodiment, the sensor is a heart pacing device.

In any embodiment, the method comprises implanting a controller for the blood pressure device in the subject, typically sub-dermally implanted.

In any embodiment, the method comprises implanting a power unit for the blood pressure device in the subject, typically sub-dermally.

In any embodiment, the power unit and controller (and optionally the circuitry) are contained in a single implantable unit, wherein the method comprises implanting the single unit, typically sub-dermally.

In any embodiment, the method comprises:
sensing, by the heart rate sensor, the heart rate of the subject; and
activating and deactivating, by the controller, the blood pumping device based on sensed heart rate data received from the heart rate sensor.

In any embodiment, the method comprises:
sensing, by the blood pressure parameter sensor, a blood pressure parameter of the subject's heart; and
modulating, by the controller, the pumping speed of the blood pumping device based on sensed blood pressure parameter data received from the blood pressure sensor.

In any embodiment, the method comprises connecting the power unit to the blood pumping device with a power lead.

In any embodiment, the method comprises:
delivering the blood pumping device percutaneously to the left ventricle;
capturing a proximal end of a power lead stowed in the blood pumping device typically in a spooled or wound configuration; and
delivering the proximal end of the power lead to the power unit.

In any embodiment, the method comprises retracting a proximal end of the power lead to the right atrium. This may be performed by the access sheath or another form of catheter device.

In any embodiment, the proximal end of the power lead is delivered to the power unit at least partially percutaneously.

In any embodiment, the power lead is delivered via the right atrium, superior vena cava and internal jugular vein/subclavian vein.

In any embodiment, the method comprises coupling the sensor and the controller (or single unit) with a sensor lead.

In any embodiment, the method comprises:
advancing an access sheath percutaneously along a guidewire so that a distal end of the access sheath is disposed within the left ventricle;
advancing the blood pumping device along a lumen of the access sheath and beyond a distal end of the access sheath;
deploying an anchoring module to anchor the blood pumping device to a wall of the left ventricle.

In any embodiment, the method comprises a step of the anchoring module self-deploying.

In any embodiment, the method comprises delivering an anchoring module to the left ventricle and coupling the blood pumping device to the anchoring module in the left ventricle.

In any embodiment, the method comprises deploying the anchoring module to anchor the anchoring module to the wall of the left ventricle and coupling the blood pressure device to the anchoring module.

Other aspects and preferred embodiments of the invention are defined and described in the other claims set out below.

### BRIEF DESCRIPTION OF THE FIGURES

FIG. 1 is a perspective view of a blood pumping device forming part of a system of the invention viewed from a blood inlet side of the device housing.

FIG. 2 is a perspective view of the heart pumping device of FIG. 1 viewed from a blood outlet side of the device housing.

FIG. 3 is a sectional view of the blood pumping device of FIG. 1.

FIG. 4 is a sectional illustration of a blood pumping device of FIG. 1.

FIG. 5 is a perspective view of a rotor forming part of the blood pumping device of FIG. 1.

FIG. 6A is a perspective view of a swept vane impeller forming part of the rotor of FIG. 5.

FIG. 6B is an end elevational view of a swept vane impeller forming part of the rotor of FIG. 5.

FIG. 6C is a side elevational view of a swept vane impeller forming part of the rotor of FIG. 5.

FIG. 7 is an illustration of a human heart with a system of the invention implanted in the left ventricle and anchored to an apex of the left ventricle.

FIG. 8 is an exploded view of the left ventricle of FIG. 7 showing the blood pumping device in-situ in the left ventricle with the inlet facing the mitral valve and showing how the device pulls blood into the LV from the RA when activated during diastole.

FIG. 9 is an illustration of one embodiment of the system of the invention in which the controller is located externally of the heart and is electrically connected to the blood pumping device by a control wire and electrically connected to an ECG sensor placed on the subject's chest.

FIG. 10 is an illustration of another embodiment of the system of the invention in which the controller is located externally of the heart and is electrically connected to the blood pumping device by a control wire and electrically connected to a heart rate sensor implanted on an external wall of the left ventricle.

FIG. 11 is an illustration of another embodiment of the system of the invention in which the controller is located externally of the heart and is electrically connected to the blood pumping device by a wireless transmitter/receiver and in which the sensor is coupled to the blood pumping device and in contact with an internal wall of the left ventricle.

FIG. 12 shows a block diagram of one embodiment of the main components of the system of the invention deployed for use in the heart.

FIG. 13 shows a block diagram of another embodiment of the main components of the system of the invention deployed for use in the heart.

FIG. 14: A graph of impeller tip diameter vs rotor speed. The speed at which the minimum impeller diameter falls within our maximum diameter range is indicated at 38348 rpm.

FIG. 15: A semi log plot of pump flow parameters with respect to rotor speed. The minimum required rotor speed based on our maximum impeller diameter is indicated at 38348 rpm.

FIG. 16: A graph of the pump coefficients with respect to specific speed. The minimum specific speed based on our maximum diameter is indicated at 1398.5.

FIG. 17: A graph of NPSHR and Head vs specific speed for an assumed impeller tip speed (u=13.272 m min⁻¹).

FIG. 18 illustrates a second embodiment of blood pumping device forming part of the system of the invention.

FIG. 19 illustrates the blood pumping device of FIG. 18 anchored in the left ventricle of a subject's heart.

FIG. 20 illustrates a second embodiment of blood pumping device forming part of the system of the invention, the device having a fluidic conduit providing fluidic communication between the left atrium and fluid inlet of the blood pumping device.

FIG. 21 illustrates the blood pumping device of FIG. 20 anchored in the left ventricle of a subject's heart.

FIG. 22A illustrates a distal end of the blood pumping device forming part of the invention, showing the power/control lead wound up within a chamber in the proximal end of the device.

FIG. 22B illustrates a retrieval catheter capturing a loop on a proximal end of the power/control lead and withdrawing the lead.

FIG. 22C illustrates the power/control lead fully retracted from the chamber of the blood pumping device and the proximal end of the lead operatively coupled to a controller/power unit.

FIG. 23 illustrates a system of the invention comprising a catheter delivery system delivering a blood pumping device to a left ventricle of a subject's heart via the femoral artery.

FIG. 24 illustrates a retrieval catheter being deployed to retrieve the proximal end of the power lead via the subclavian vein and superior vena cava.

FIG. 25 illustrates the blood pumping device in the left ventricle coupled to the power unit/controller which is implanted sub dermally in the chest via the power lead which extends through the subclavian vein, superior vena cava, right atrium, interatrial septum, left atrium and into the left ventricle.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

Additionally, the terms “treatment” or “treating” refers to an intervention (e.g., the use of the device of the invention to assist systole of the left ventricle) which prevents or delays the onset or progression of a disease or reduces (or eradicates) its incidence within a treated population. In this case, the term treatment is used synonymously with the term “prophylaxis”.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

“Blood suction device” means a device configured for implantation in a chamber of the heart, particularly the left ventricle, that upon activation can draw blood into the left ventricle from the right atrium through the mitral valve during diastole, thus assisting the left ventricle fill with blood. The device generally comprises an impeller. The impeller typically comprises a rotor with vanes disposed within a housing with a blood inlet and a blood outlet. The housing is generally tubular and elongated wherein the blood inlet is disposed at one end or the middle of the housing and the blood outlet is disposed at one end of the housing. The impeller is mounted for rotation within the housing with the vanes disposed towards the blood outlet of the housing. In use, the device is typically anchored to a wall of the left ventricle such that the blood inlet of the housing is positioned closer to the mitral valve than the blood outlet of the housing. The housing typically has a longitudinal axis that in use is directed towards the mitral valve. The device generally includes a motor, which may be an electromagnetic motor comprising a stator. The device may also include the controller and a sensor. The device may include or be operatively coupled to an inductive charging apparatus.

“Output parameters” as applied to the blood suction device include the frequency of activation of the device and the pump flow pressure (e.g., impeller rotational speed during activation. It will be appreciated that during phases of deactivation of the blood suction device that the impeller rotation may be maintained but at a rotational speed that is significant reduced compared with an activation phase. Thus, for example the impeller may rotate at a rotational speed of 50% or less than the activation phase rotational speed.

“Activation during ventricular diastole and deactivation during ventricular systole” mans that the blood pumping device is activated during at least a part of ventricular diastole (usually at least 50%, 60%, 70%, 80%, 90% or 100% of ventricular diastole) and the blood pumping device is deactivated during ventricular systole. For example, the blood pumping device may be deactivated when system detects closure of the aortic valve (during ventricular systole) and may be activated when the system the opening of the aortic valve (during ventricular filling and subsequent atrial contraction). The blood pumping device may be activated and deactivated at a frequency based on the frequency of the heartbeat detected by, for example, a heart pacing sensor.

“Anchor” means an element configured for engaging tissue, for example a wall, septum or apex of the left ventricle of the heart. The anchoring assembly may comprise one, two, three or more anchors. One may attach to the left ventricular septum, and one may attach to the apex of the left ventricle. The anchoring assembly comprises anchors that are configured to be deployed and embedded in the tissue of the wall of the left ventricle to ensure a secure attachment of the blood suction device. Anchoring is usually at a minimum of two points in the wall. The anchors are adapted to withstand repeated loads generated by both the heart suction device, as well as loads generated by the tissue in which they are embedded. In addition, the anchors are designed to minimise damage to the surrounding tissue, through designs that minimise the damage caused through penetration of the anchor through muscle tissue, and any subsequent damage to the tissue generated by repeated and continuous contraction cycles. The anchor may be formed from any suitable structure. Anchors are selected according to tissue type and the condition of the tissue. In one embodiment, the anchor is encapsulated with an elastic polymer membrane that expands circumferentially when the anchoring assembly is compressed at deployment configuration. In one embodiment, the coupling between at least one of the anchors and the blood suction device comprises a magnet or magnetisable element. This facilitates coupling of the anchor and the blood suction device at a target location in-vivo. In one embodiment, the anchors are configured for detachable coupling with the heart suction device. This allows one or both anchors to be delivered to the target location separately from the heart suction device. In one embodiment, the device is delivered in two parts, a first part comprising a first anchor, and a second part comprising the heart suction device coupled to the second anchor.

The system of the invention may include an inductive charging apparatus. “Inductive charging apparatus” means an apparatus for charging the system, or any device of the system, wirelessly by using any one of electromagnetic field, wireless radio waves or magnetic resonance charging to transfer energy to charge the device. For example, the blood suction device may comprise an inductive charging apparatus.

The system of the invention is designed to deliver clinically effective functional support to the left ventricle of the heart during the filling phase (diastole). The system increases the flow of blood into the left ventricle of the heart during diastole in which it is implanted and then is deactivated during systole. In one embodiment, the blood pumping device is deactivated during all of ventricular systole. The controller is programmable such that the system can act as an auto-adaptive system that responds to physiologic cues. The system can continuously adapt to specific user requirements. The blood suction device is configured by means of the controller to activate in a pattern synergistic to the natural cycle of the heart to, for example, activate the blood suction device during diastole and deactivate the blood suction device during systole. Thus, as the heart rate of a subject increases, the frequency of activation and deactivation of the blood suction device changes in sync with the heart rate based on signals received from the sensor. In addition, the controller can modify the impeller rotation speed based on physiological cues received from the sensor, for example increase the impeller rotational speed during periods of elevated heartbeat and reduce the impeller rotational speed during periods of reduced heart rate. The controller may comprise a computation device configured to receive data from the sensor, compare the stored with stored reference data, and modify the output parameters of the blood suction device based on the comparison.

The controller may be configured for use outside the heart. Thus, the system may include a control lead that operatively couples the heart suction device implanted within the heart with the controller located outside the body. The control lead may extend through a wall of the heart and through the chest to the external controller. In another embodiment, the control lead may extend percutaneously from the left ventricle through part of the vasculature and out of the body at a suitable location. The controller may be configured to be wearable by a subject. The controller may be connected to a power source, for example a battery. In another embodiment, the controller may be configured for implantation within the heart, as part of the blood suction device, and be configured to receive data from the sensor.

The sensor may be part of the system of the invention, or the system of the invention may be configured for use with a separate sensor. For example, the system may include a sensor configured for implantation in the left ventricle of the heart where it is operatively connected or coupled to the blood suction device. This may be a wire/sensor element configured to extend into contact with a wall of the heart for sensing a parameter of heart contraction/relaxation or configured to contact blood in the left ventricle or another chamber to detect a parameter of the blood (for example pressure or flow rate).

In another embodiment, the sensor is configured for implantation on an external wall of the heart and operatively coupled to the controller, which may be mounted externally of the subject's body.

In any embodiment, the system comprises a graphical user interface which may form part of the controller. The controller may be configured to display on the GUI data relating to the functioning of the system, for example heart rate, blood pressure, frequency of activation of the impeller, rotational speed of the impeller.

In any embodiment, the sensor is configured to detect ejection fraction of the left ventricle. In any embodiment, the controller is configured to compare the detected ejection fraction of the left ventricle with one or more ejection fraction reference values and modify the output parameters of the blood suction device based on the comparison. In any embodiment, the controller is configured to switch off the blood suction device for a period of time when the ejection fraction is detected to be reduced. In any embodiment, the controller is configured to resume operation of the blood suction device when the ejection fraction is detected to be preserved.

The system may further include telemetric components for transmitting and receiving signals relating to the activity of the left ventricle, heart or the activity of the system. Telemetric components may include wireless medical telemetric elements using radio frequency to relay data such as pulse, heart rate, and electrical activity of the heart to the controller.

The controller may be configured to control the operation of the blood suction device either through automatic execution of program instructions in memory and/or upon receiving an external input from a user. The memory component of the system may include ROM and RAM memory. The controller may take the form of a microprocessor.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

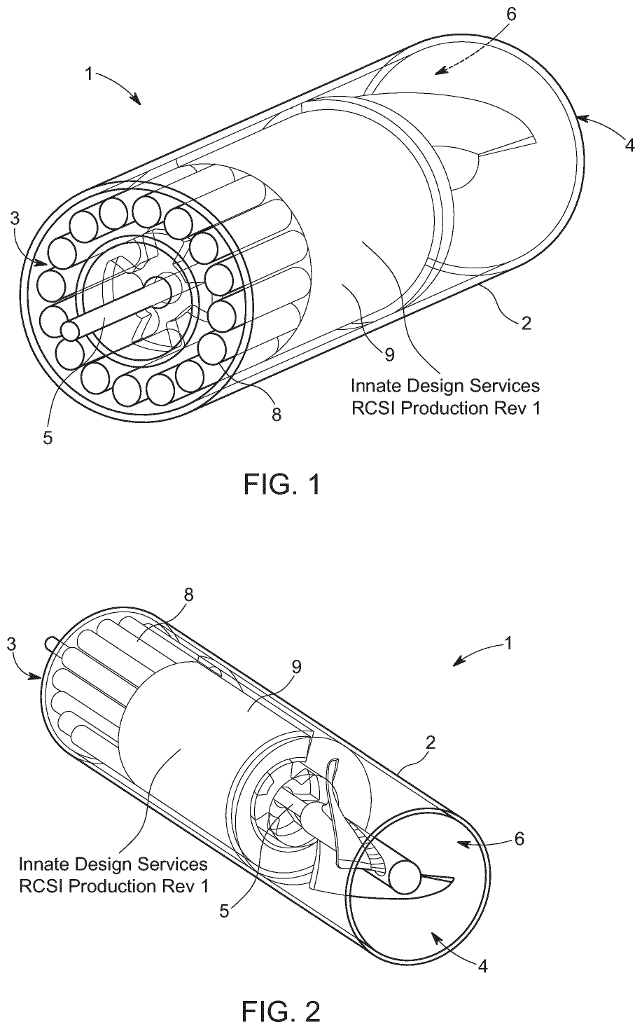

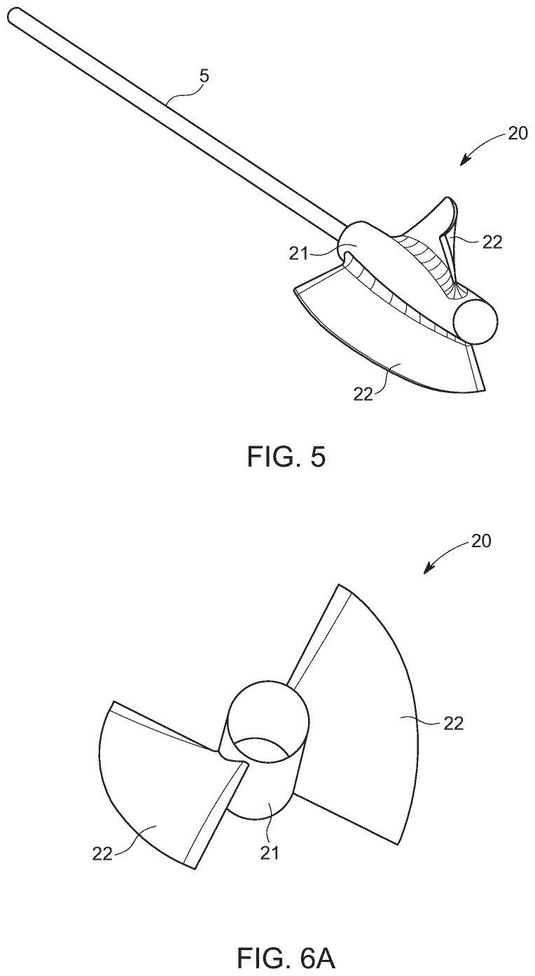

Referring to the drawings and initially to FIGS. 1 to 6, there is illustrated a blood suction device forming part of a system of the invention and indicated generally the reference numeral 1. The device 1 comprises an external tubular housing 2 with a blood inlet 3, blood outlet 4, and internal lumen 6 extending between the inlet 3 and outlet 4. The housing has a length of 30 mm and a diameter of 7.6 mm. A rotor 5 is mounted on spaced apart bearings 7 and extends axially through the internal lumen 6 for rotation within the lumen. A stator 8 is mounted within the housing adjacent the blood inlet 3 surrounding a proximal end of the rotor and in use functions to rotate the rotor during activation of the blood pumping device. A PCB 9 comprising electronic circuitry for the operation of the device is disposed in a central part of the housing surrounding the rotor 5.

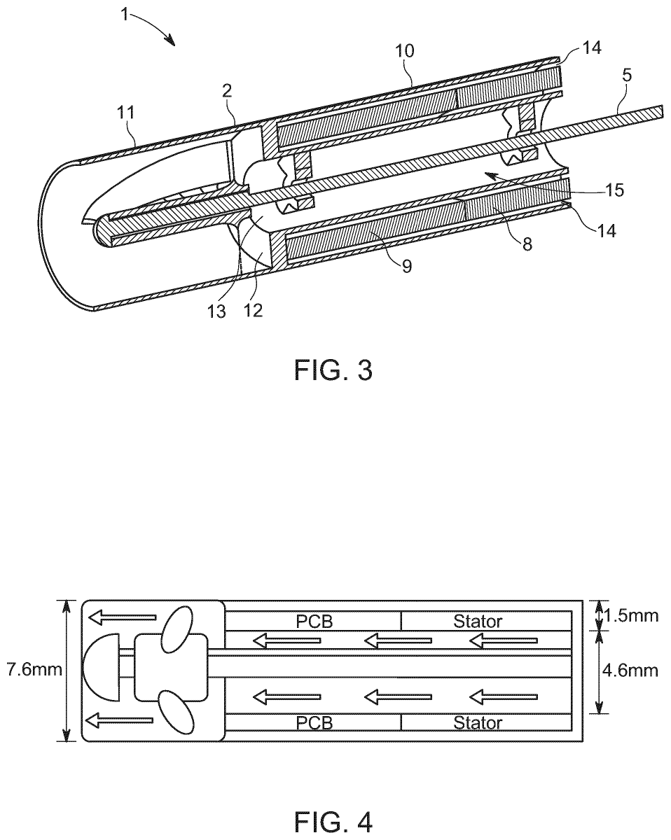

Referring to FIG. 3, the housing 2 has a proximal part 10 and distal part 11 separated by a dividing wall 12 with a central annulus 13. The proximal part of the housing comprises an annular compartment 14 that contains the stator 8 and PCB 9 and a central lumen 15 providing a passageway for blood during activation of the device. The PCB 9 includes the following components: resistor, diode, transistor, microprocessor, pressure sensors.

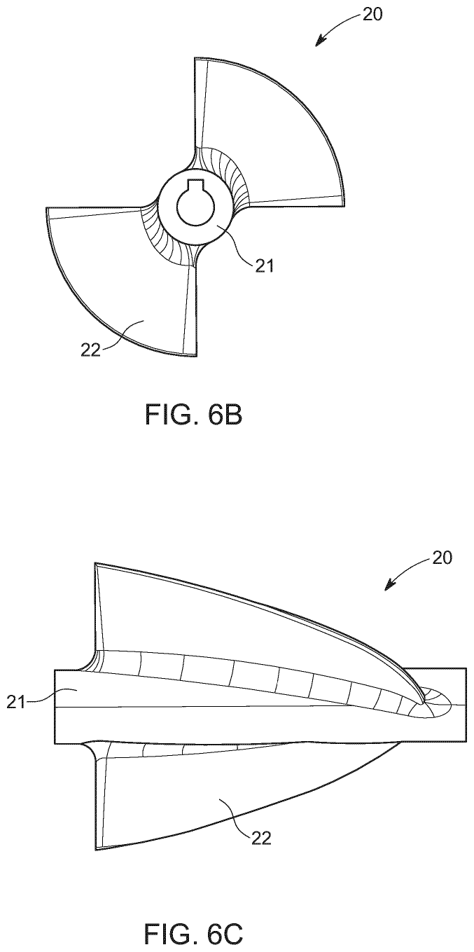

Referring specifically to FIGS. 4 to 6, an impeller device 20 is mounted to a distal end of the rotor 5 and comprises a tubular hub 21 with a diameter of 1.665 mm and two elongated swept vanes 22 attached to opposite sides of the hub in a symmetrical manner. Each vane has an axial length of 13 mm and a radial width of 4.995 mm and sweeps around the hub along its length from a distal end to a proximal end at an angle of about 100° (sweep angle) illustrated as ∅ in FIG. 6B. The clearance between each vane and the surrounding external housing is 0.5 mm.

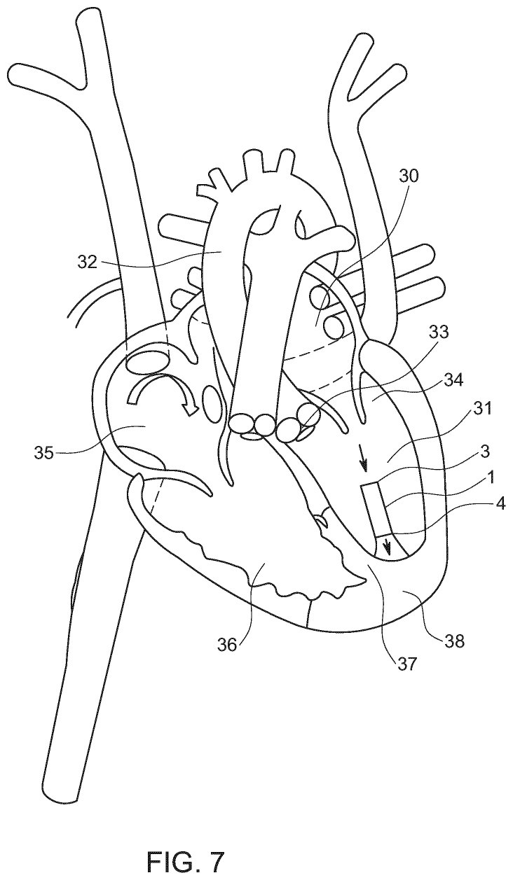

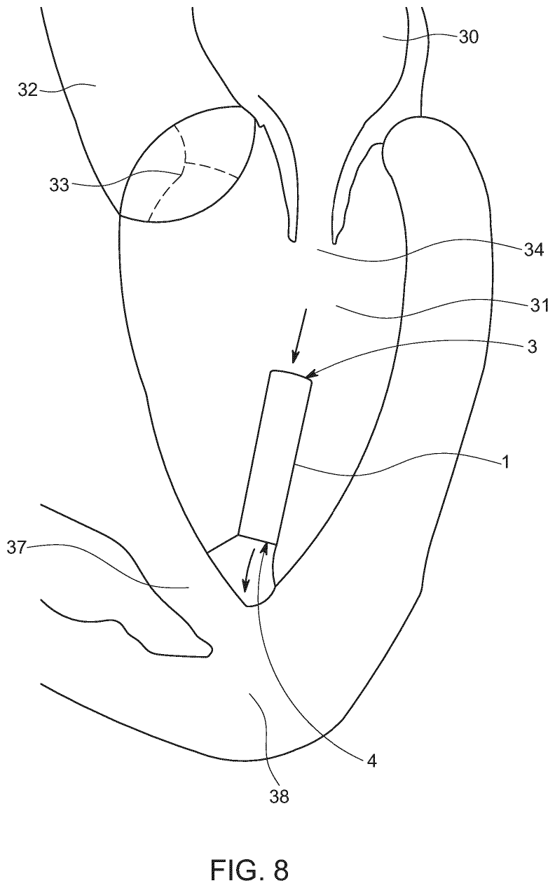

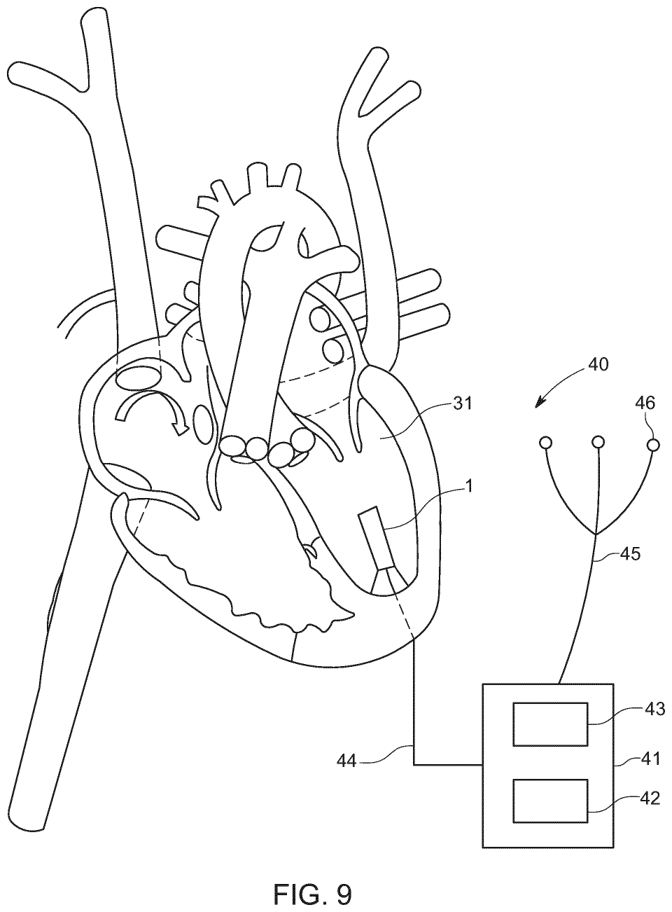

FIGS. 7 and 8 illustrate a human heart including left atrium 30, left ventricle 31, aorta 32 and aortic cusps 33, mitral valve 34, right atrium 35, right ventricle 36, left ventricular septum 37 and left ventricular apex 38. The blood suction device 1 is shown implanted in the left ventricle with two anchoring arms, a first anchoring arm 39A anchored in the lower part of the left ventricular septum 37 and a second anchoring arm 39B anchored to the apex 38. The device is positioned such that the blood inlet 3 of the device 1 faces towards the mitral valve 34 and left atrium and the blood outlet 4 faces the apex of the left ventricle. Upon activation of the device during diastole, blood is drawn into the device through the inlet 3 and ejected through the outlet 4 as illustrated by the arrows A, which assists the emptying of blood from the right atrium into the left ventricle. Once diastole is completed, the device is deactivated as the mitral valve closes and the aortic cusps open, and the left ventricle contracts to pump blood into the aorta unassisted.

FIGS. 9 to 11 illustrate some embodiments of the system of the invention, in which parts described with reference to the previous embodiments are assigned the same reference numerals.

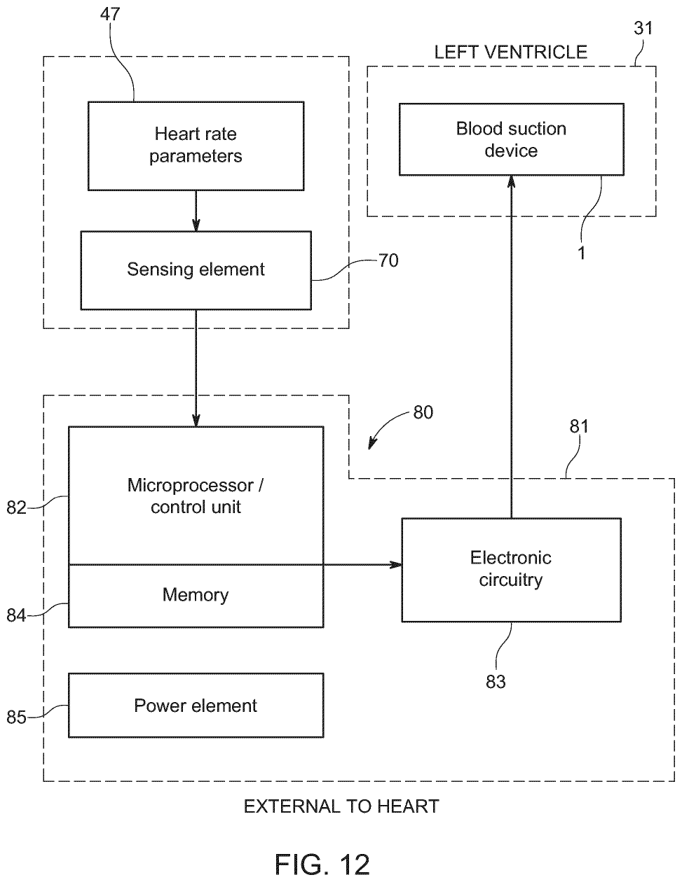

In FIG. 9, a system of the invention 40 comprises a blood suction device 1 shown implanted in the LV of the heart as described previously and a controller device 41 external to the subject's body containing a power module 42 and a controller/processing device 43. A control lead 44 extends from the LV through a wall of the LV and operatively connects with the controller device 41. A sensing lead 45 operatively connects the controller device with an ECG sensor 46 mounted to the subject's chest. The ECG array has one or more leads that are placed on the skin of the user, as shown in FIG. 12. The ECG leads may be positioned subcutaneously or implanted, such as for example in the left parasternal region of the body. In this embodiment, the ECG monitors the electrical parameters of the subject heart, the controller receives the electrical parameters and activates and deactivates the blood pumping device 1 in a pattern synergistic with the electrical parameters to activate the blood suction device during diastole and deactivate the blood suction device during systole to assist the LV ventricle fill with blood during diastole. The controller also controls other parameters of the blood suction device based on data received from the sensor, including increasing the impeller rotational speed during diastole during periods of activity (e.g., when the heartbeat rises above a resting heart rate) and lowering the impeller rotational speed during diastole during periods of rest.

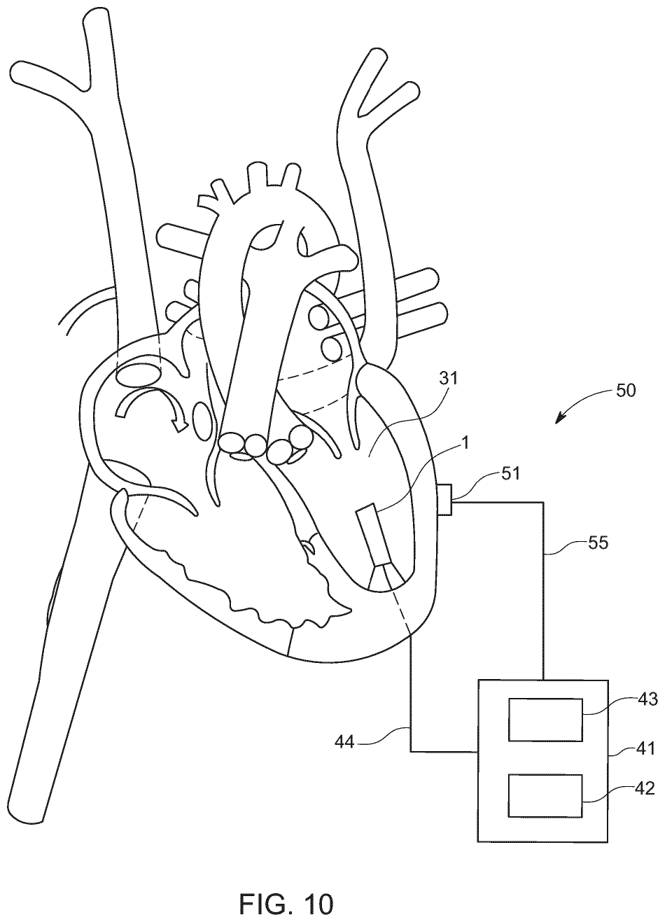

FIG. 10 illustrates an alternative embodiment of the system of the invention, indicated by the reference numeral 50 in which parts described with reference to the previous embodiments are assigned the same reference numerals. In this embodiment, a sensor 51 to detect left ventricle contraction parameters is implanted in the subject's chest by means of keyhole surgery and anchored using a suitable anchoring means on an external wall of the left ventricle. A sensor lead 55 extends between the sensor 51 and the controller device 41 to provide data comprising left ventricle contraction parameters to the controller. The use of the system to augment the filling of the left ventricle during diastole is the same as that described previously with reference to FIG. 9.

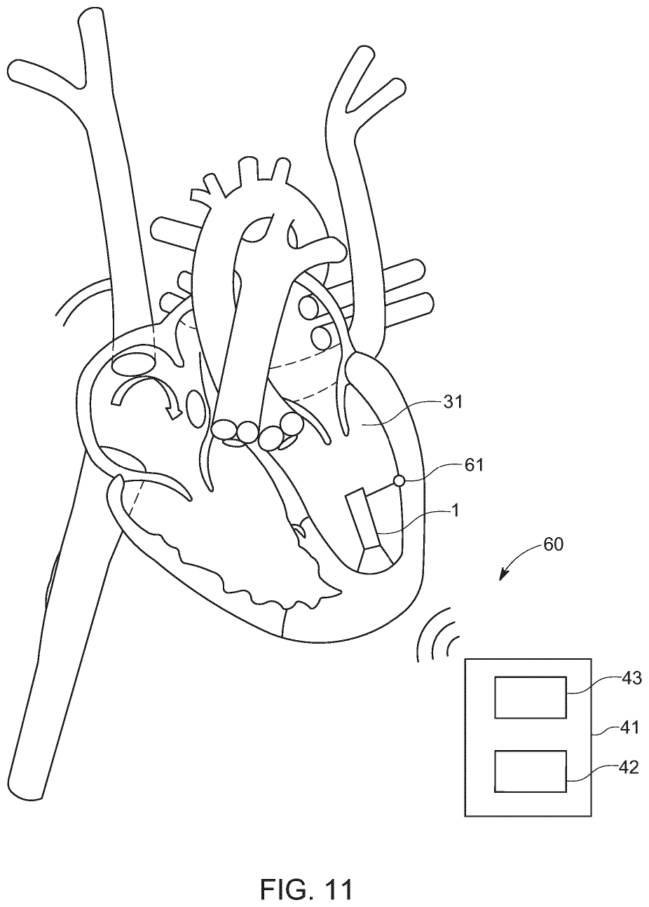

FIG. 11 illustrates an alternative embodiment of the system of the invention, indicated by the reference numeral 60 in which parts described with reference to the previous embodiments are assigned the same reference numerals. In this embodiment, a sensor 61 is provided in the form of a wire connected to the housing of the blood suction device 1 configured to deploy upon deployment of the device in the left ventricle into contact with a wall of the left ventricle. The wire is configured to detect electrical parameters of the heart and relay the detected electrical parameters to a wireless transmitter/receiver mounted in the housing of the blood suction device. The controller includes a wireless receiver/transmitter to receive the data from the sensor 61 and to relate output parameters to the blood suction device. The use of the system to augment the filling of the left ventricle during diastole is the same as that described previously with reference to FIG. 9.

FIG. 12 shows a block diagram of one embodiment of the main components of the system of the invention. In the embodiment shown in this figure, the blood suction device 1 comprising an impeller pump is anchored in the left ventricle 31 of the subject's heart. A sensor 70 configured to detect a heart rate parameter is located outside of the heat. The sensor may be an ECG sensor comprising ECG leads disposed on the subject's chest, or another type of heart rate parameter sensor located externally of the subject's heart. The sensor 70 is configured to detect a heart rate parameter 47, in this case left ventricle contraction parameters.

The sensor data output from the sensor 70 is communicated to the microprocessor/control unit 81 by means of either wired or wireless communications. The microprocessor/control unit 81 is configured to continuously assess the time of diastole and systole based on the received sensor data and optimally synchronise the activation and deactivation of the blood suction device 1 into sync with the diastole and systole stages of the cardiac cycle.

In use, the sensor 70 detects the heart rate parameters of the heart and sends the sensor data to the microprocessor/control unit 81. The received sensor data is analysed by the microprocessor/control unit 81 according to the instructions stored in the memory 84. The microprocessor/control unit 81 then delivers control signals to the power unit 85 and to the electronic circuitry 83 so as to modify the output parameters of the heart suction device 1 as appropriate based on the sensor data so as to activate the heart suction device 1 in a pattern synergistic to the natural contraction cycle of the heart to activate the device during diastole and deactivate the device during systole.

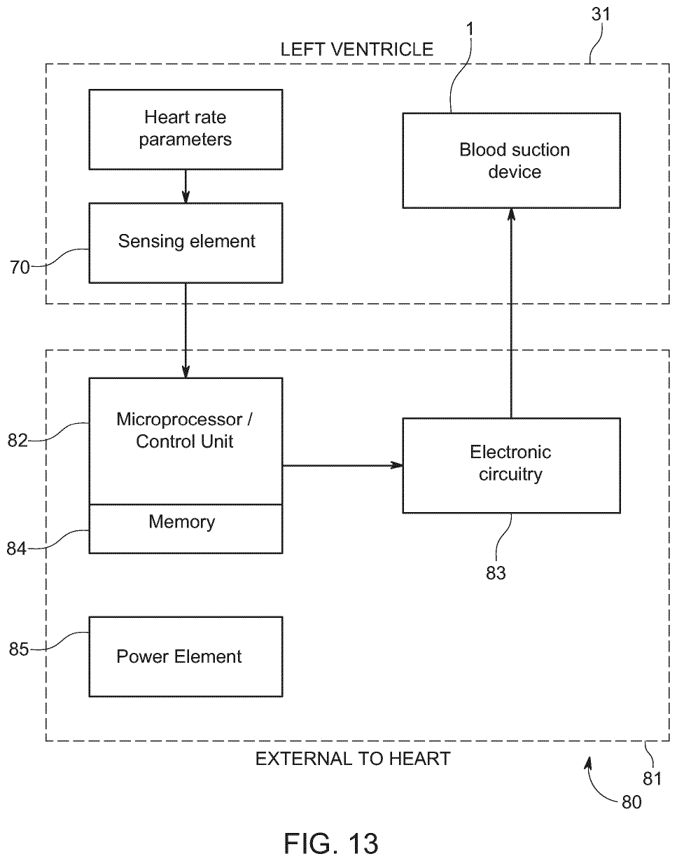

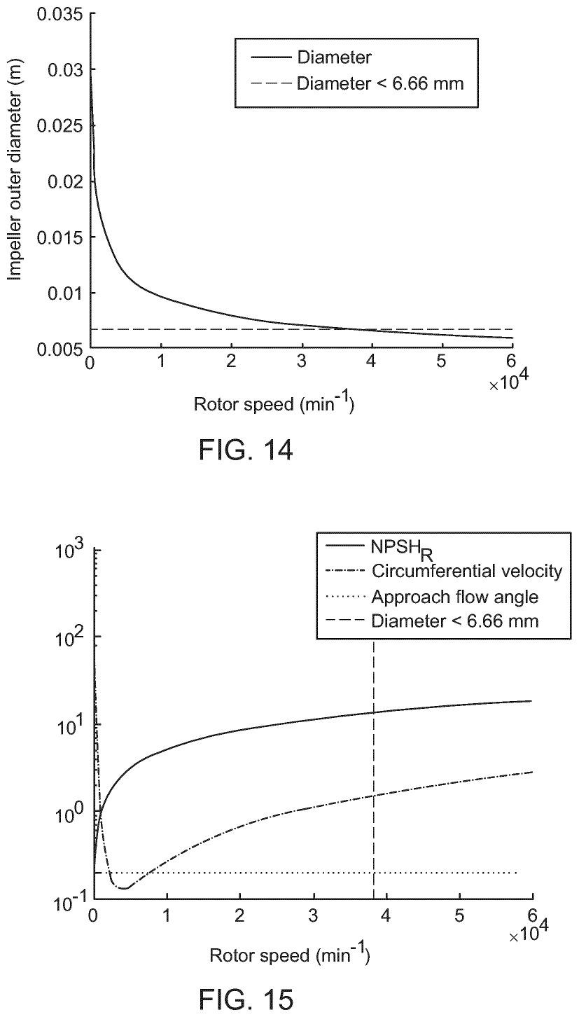

FIG. 13 shows a block diagram of another embodiment of the main components of the system of the invention. In this embodiment, which is substantially the same as the system described with reference to FIG. 11, the sensor 70 for sensing a heart rate parameter is located within the left ventricle of the heart and sends sensed data to the external microprocessor/control unit 81 by means of wired or wireless communication. The use of this embodiment of the system is substantially the same as that described previously with reference to FIG. 11.

### Delivery of the Blood Suction Device to the LV

The device may be delivered percutaneously via femoral artery/subclavian artery or femoral vein/subclavian vein. The device may also be delivered through transapical route, whereby an incision is given in an intercostal space and the device is delivered through the apex.

### Supporting Data

FIG. 4 illustrates a blood suction device according to one embodiment of the invention. The embodiment of FIG. 4 is designed to be suitable for delivery in a 29 Fr (9.66 mm OD) catheter to give more space for internal components such as a printed circuit board and stator coils. The wall thickness is customisable to a certain extent, but we will assume a wall thickness of 1 mm. The internal diameter is therefore 7.66 mm. Assume 0.5 mm for clearance and wall thickness, our impeller tip diameter cannot exceed 6.6 mm.

The pump parameters are based on a series of linked equations found in Gülich (2020—Page 455) [Gülich, J., 2020. Centrifugal Pumps. 4th ed. Springer International Publishing, p.455.]

The quantities already known are Flow rate (Qopt), Head (Hopt), Net Positive Suction Head Available (NPSHA), Cavitation Safety Factor (FNPSH), and Pump Efficiency (ηh). The values and any calculations described are included in the MATLAB script Pump_graph.m.

The flow rate required by the pump is equal to the volume of blood the pump needs to move in one heartbeat multiplied by the heart rate, expressed in m3 s-1.

Assuming 20 ml of blood must be moved and a worst-case heart rate of 200 bpm; Qopt=6.66×10-5 m3 s-1.

The required head is the pressure reduction necessary to alleviate LA hypertension in HFpEF. This is estimated to be 10 mm Hg in the worst-case scenario. This pressure can be expressed in metres by dividing by the specific weight of the fluid (assumed to be the same as water); Hopt=0.1359 m.

The Net Positive Suction Head Available is equal to the net pressure at the inlet of the pump minus the vapour pressure of the fluid expressed in metres. The total pressure in the LA is taken as the sum of the LAP at diastole (20 mm Hg—this is when the pump will be active), the height of the LA (3 cm), and atmospheric pressure (760 mm Hg). The vapour pressure of blood is assumed equal to that of water (47 mm Hg). NPSHA=9.995 m.

The cavitation safety factor is estimated to limit the amount of cavitation by ensuring the pressure does not drop below the blood vapour pressure, this is estimated as FNPSH=0.8*NPSHA.

The pump efficiency cannot be calculated and must be derived experimentally. For axial flow pumps typically ηh=85%.

The desired quantities here are the rotor speed (n), the impeller tip diameter (d2), the hub ratio (v), the specific speed (nq), the pressure coefficient (ψ), the flow coefficient (φ), and the cavitation coefficient (σ).

The quantities can be calculated iteratively, but in practice it is simpler to use a MATLAB script to plot the quantities with increasing rotor speed and determine the possible pump characteristics based on the generated graphs of these parameters.

FIG. 15 shows the calculated minimum impeller diameter for a range of rotor speeds. By selecting a value of diameter less than or equal to our maximum impeller diameter (6.66 mm) we can determine the minimum rotor speed required for the pump to be 38348 rpm.

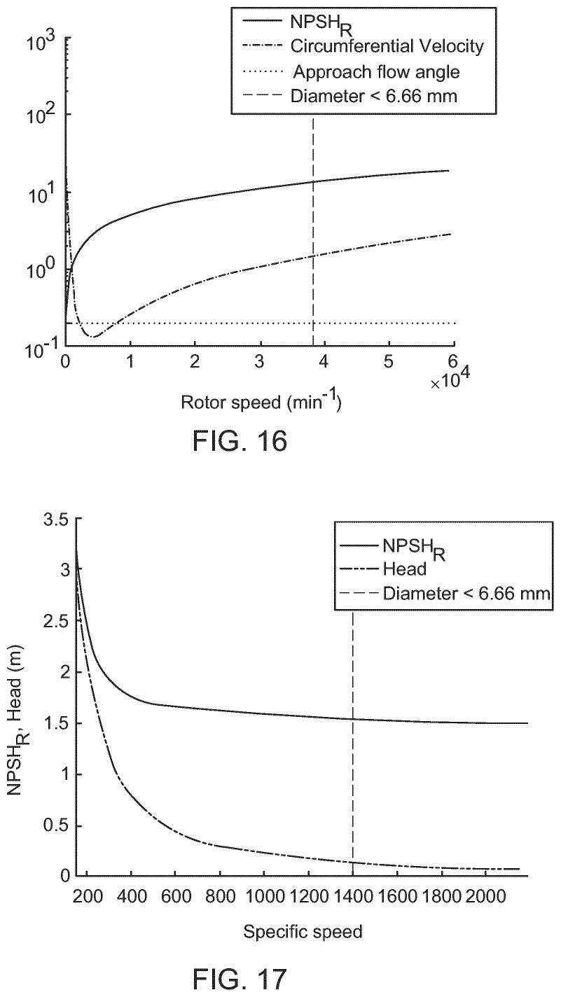

FIG. 16 is a semi log graph of the flow parameters with respect to rotor speed. Again, the minimum required rotor speed is indicated at 38348 rpm. This graph indicates that the parameters at the required rotor speed are: NPSHR=1.5322 m, circumferential velocity u=13.272 m min^{-1,} and approach flow angle β=0.1455°.

FIG. 17 shows the pump coefficients with respect to specific speed. Here the minimum required rotor speed has been converted to specific speed and indicated on the graph at 1398.5. The values of the coefficients can be evaluated at this point on the graph as: Ψ=0.0151, φ=0.1465, v=0.0608, and σ=0.1708.

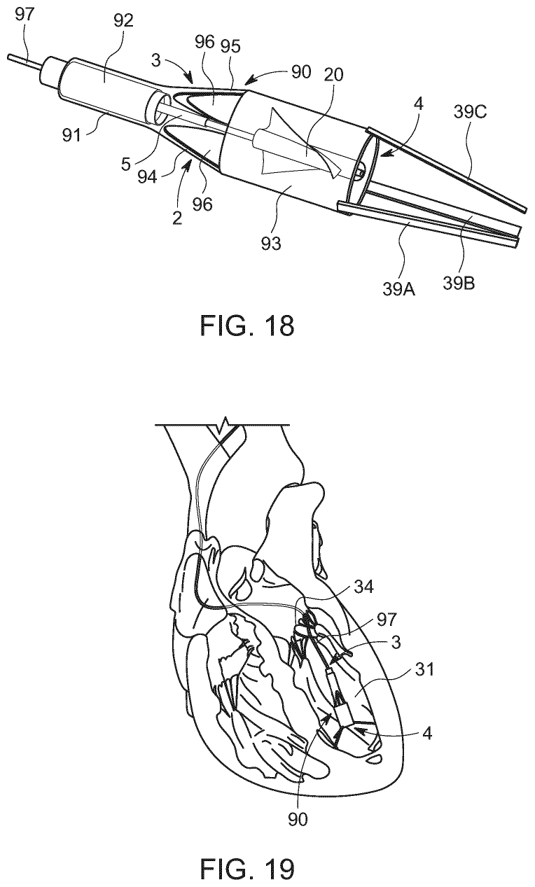

FIG. 18 shows NPSHR and pump head vs specific speed when the impeller tip speed is set to the calculated circumferential velocity evaluated previously (u=13.272 m min-1). The required specific speed is indicated at 1398.5 and evaluating NPSHR and head at this value yields 1.5332 m and 0.1360 m respectively. This value for NSPHR is the same as evaluated from FIG. 4 and the value for head is the same as specified as a requirement for the pump.

FIG. 7 shows a table taken from Gülich (2020) pg. 456 which outlines the number of impeller blades and diffuser vanes required for ranges of specific speeds. It can be seen from the table that because our specific speed>290, the number of impeller blades should be 2 and the number of diffuser vanes should be 5/7/9. Rather than work through the calculations manually for the blade profiles, a software tool called CFTurbo (CFturbo GmbH, Germany) was used to finalise blade design. This tool takes in information calculated in the previous section and semi-automates the process of impeller design. The inputs selected were as calculated for a 200 bpm HFpEF condition as stated previously. However, the hub ratio calculated for this condition was too small to be practical. Therefore, the code was run again for a range of different heart rates and a compromise hub ratio of 0.25 was selected. The final impeller design was exported and prepared for inclusion in the full CAD file.

Referring to FIGS. 18 and 19, an alternative blood pumping device forming part of a system of the invention is illustrated, in which parts described with reference to the previous embodiments are assigned the same reference numerals. The blood pumping device, illustrated generally by the reference numeral 90, comprises a housing 2 with a blood inlet 3, blood outlet 4, and a rotor 5. In this embodiment, housing comprises a cylindrical proximal part 91 comprising a motor 92 operatively coupled to a proximal end of the rotor 5, a cylindrical distal part 93 comprising the impeller 20 attached to a distal part of the rotor 5 and a distal blood outlet 4, and a central part 94 comprising the blood inlet 3. The cylindrical distal part 93 has a diameter about 2 times larger than a diameter of the cylindrical proximal part 91. The central part 94 comprises a plurality of struts 95 that couple the proximal part 91 to the distal part 93, the struts defining a plurality of large triangular blood inlet apertures 96 that function as the blood inlet. The device has a length of about 20-30 mm, and a diameter at the proximal end of 1.5 to 3 mm and at the distal end of 3 to 7 mm and is therefore suitable for percutaneous delivery to the left ventricle. A power/control lead 97 is coupled to the motor and extends proximally from the device 90 into the left atrium. The distal end of the power/control lead 97 is operatively coupled to a power unit (not shown) that is sub-dermally implanted in the subject's chest. Three anchoring arms 39A, 39B and 39C are attached to the cylindrical distal part 93 of the housing and are pivotally adjustable from a stowed delivery configuration shown in FIG. 18 to an outwardly splayed deployed configuration shown in FIG. 19. The arms are formed from nitinol and are resiliently biased into the outwardly splayed configuration and self-deploy into this deployed configuration once the device 90 is moved beyond a distal end of an access/delivery sheath (not shown). A tip of each arm includes an anchoring barb 98 to engage the tissue of the wall of the left ventricle.

In use, the device 90 is loaded into a proximal end of an access sheath with the anchoring arms constrained in the stowed delivery configuration. The device 90 is then advanced through a lumen of the sheath and into the left ventricle where it is deployed out of a distal end of the sheath resulting in the anchoring arms self-deploying to anchor the device to the wall of the left ventricle. The device is positioned such that the blood inlet 3 of the device 1 faces towards the mitral valve 34 and left atrium and the blood outlet 4 faces the apex of the left ventricle 31. Delivery of the device through an access sheath may employ an elongated delivery shaft having a distal end detachably coupled to a proximal end of the device, and where the device is advanced through the access sheath by advancing the delivery shaft into the access sheath. The access sheath and delivery shaft may be associated with a handle having an actuator to move the delivery shaft axially relative to the access sheath. Once in situ, the power/control lead 97 is coupled with a controller and power unit, and the motor is activated and deactivated in a pattern synchronous with the cardiac cycle based on cardiac cycle data received by the controller from a sensor, including activation of the motor during ventricular diastole to assist the left ventricle filling with blood, and then deactivated during ventricular systole when the left ventricle contracts to eject blood. The frequency of activation/deactivation is modulated in real time based on changes in the heartbeat of the subject as detected by the sensor. A sensor may also provide blood pressure parameter data to the controller, which can be used to control the speed of rotation of the motor during the activation phases. In this way, the system of the invention can set the speed of rotation of the impeller according to the blood pressure detected in the heart and modulate the impeller speed according to changes in blood pressure, for example when the subject's heart has to work harder due to exercise or physical exertion.

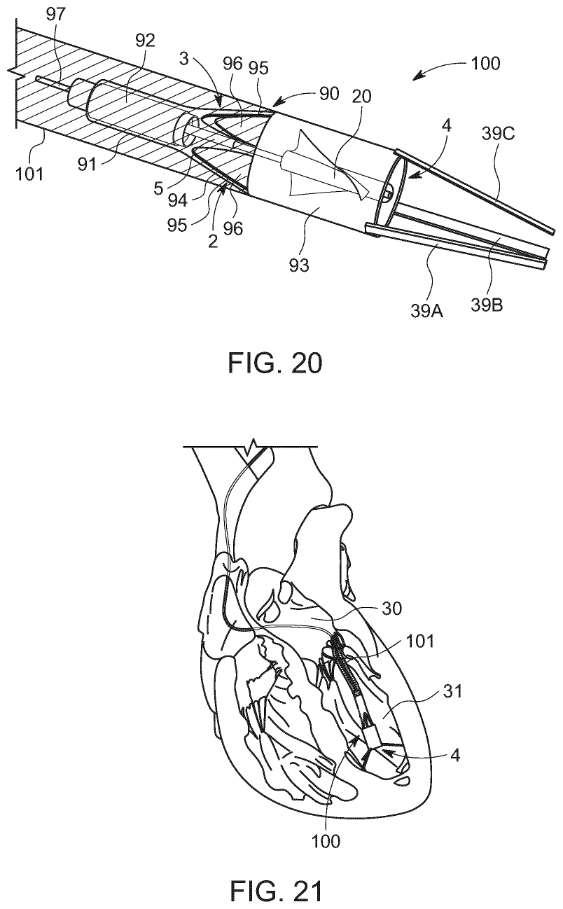

Referring to FIGS. 20 and 21, an alternative blood pumping device forming part of a system of the invention is illustrated, in which parts described with reference to the previous embodiments are assigned the same reference numerals. The blood pumping device, illustrated generally by the reference numeral 100, is the same as the device 90 described above but additionally includes a fluidic extension conduit 101 configured to provide fluidic communication from inside the left atrium 30 to the blood inlet apertures 96 of the device 90. The conduit 101 is generally flexible and comprises an open distal end 102 that fluidically mates with the proximal end of the cylindrical distal part 93 of the device housing and an open proximal end 103 to receive blood from the left atrium. The conduit 101 is dimensioned to extend partially into the left atrium 30 such that the open proximal end 103 is positioned in the left atrium. The distal end of the conduit may be configured for anchoring to the mitral valve 34. The conduit 101 may be integrally formed with the device 100 or it may be detachably coupled to the device. The use of this embodiment is the same as that described in relation to the embodiments of FIG. 18 except that blood is drawn into the device 100 through the conduit 101 directly from the left atrium.

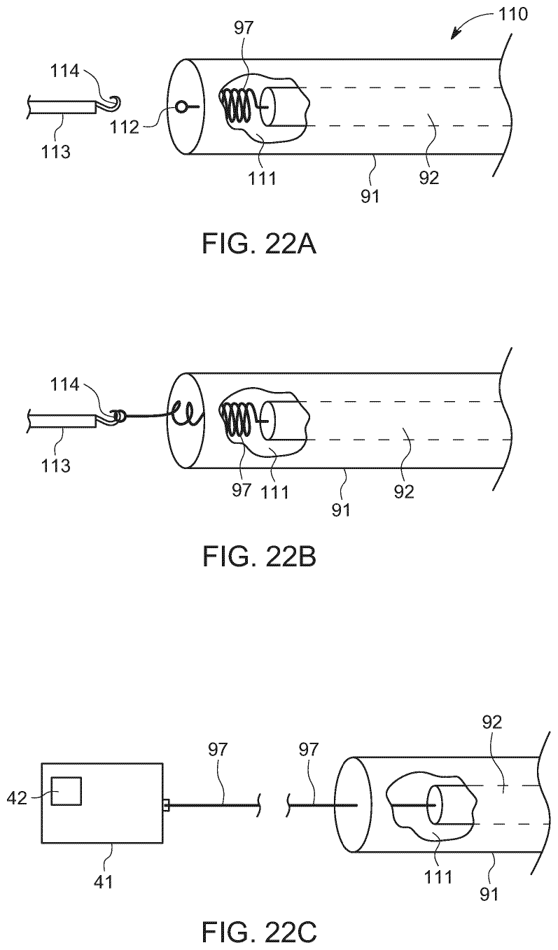

Referring to FIGS. 22A, 22B and 22C, an alternative blood pumping device forming part of a system of the invention is illustrated, in which parts described with reference to the previous embodiments are assigned the same reference numerals. The blood pumping device, illustrated generally by the reference numeral 110, is the same as the device 90 described above except that the cylindrical proximal part 91 of the device housing comprises a chamber 111 and the power/control lead 97 packed inside the chamber 111 proximally of the motor 92. A distal end of the power/control lead 97 comprises a loop 112. A retrieval catheter 113 with a distal hook 114 is also illustrated. In use, the blood pumping device 110 is delivered to the left ventricle as described previously with the power/control lead 97 spooled inside the chamber 111 (FIG. 22A). Once the device is anchored in the left ventricle, and the delivery shaft detached from the device and withdrawn, the retrieval catheter 113 is advanced through a second access sheath (not known) that extends from an entry point at the subclavian vein, through the superior vena cava, and into the left atrium via the right atrium. The retrieval catheter is advanced through the second access sheath until the distal hook 114 snags the loop 112 of the power/control lead 97 (FIG. 22B), and then the retrieval catheter is retracted to pull the lead 97 proximally through the right atrium, superior vena cava and subclavian vein, from where it can be electrically coupled with a controller 41 comprising a power unit 42 (FIG. 22C) that is implanted sub dermally in the subject's chest. The use of this embodiment of the device is the same as described previously.

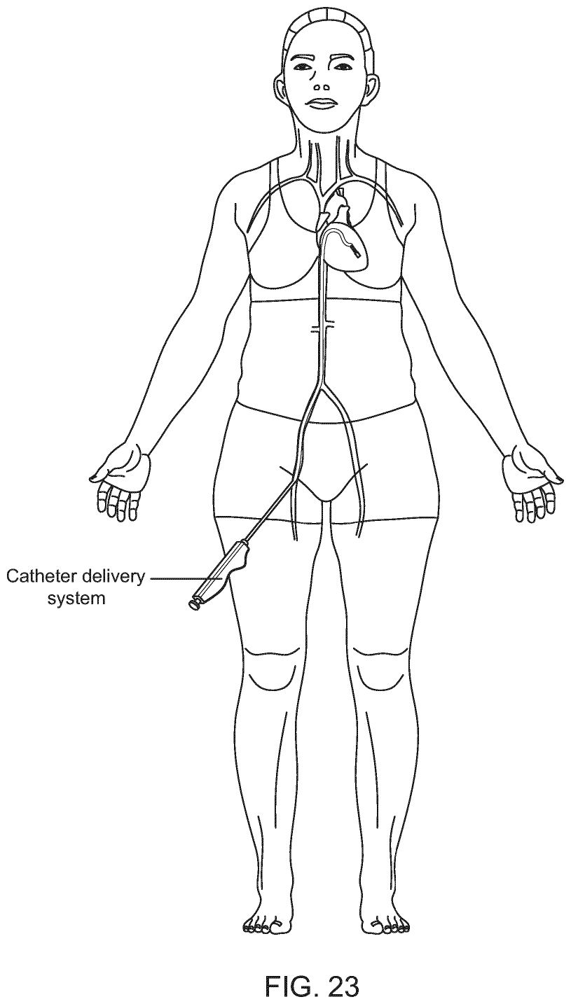

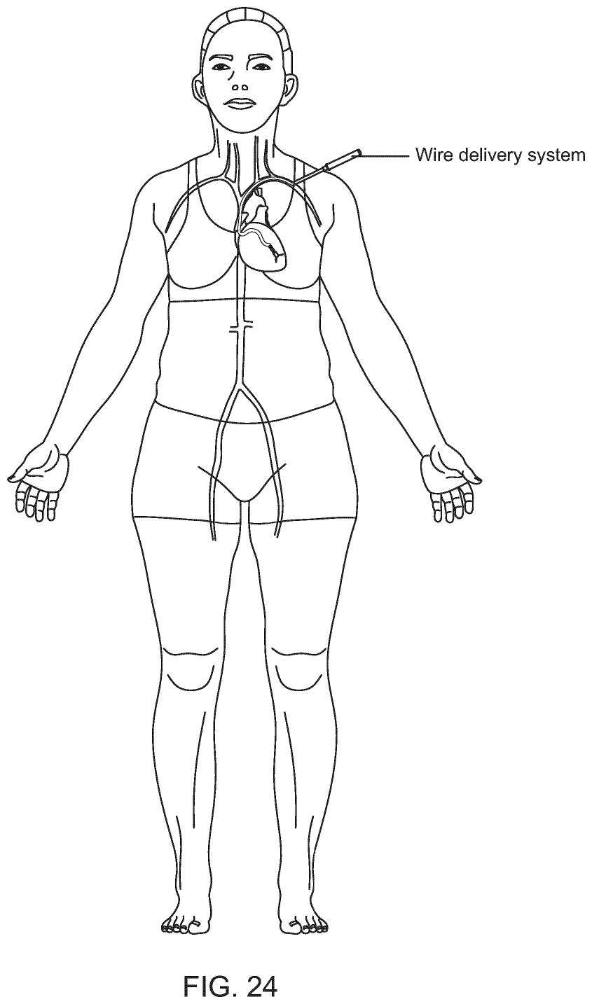

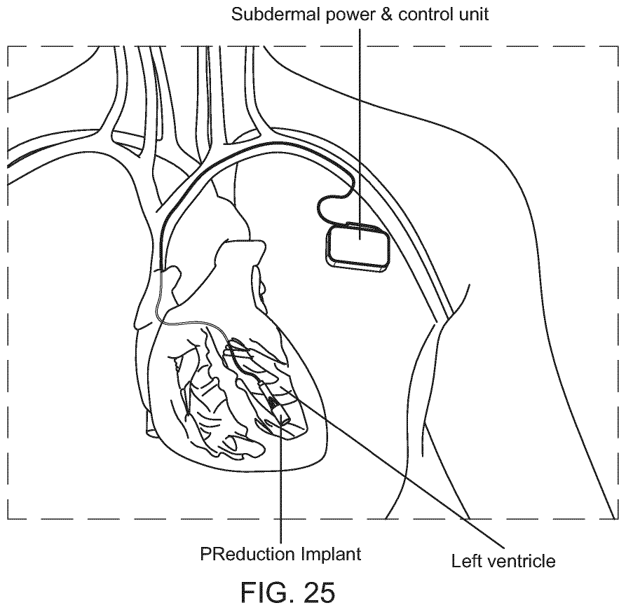

FIGS. 23 to 25 illustrate the system of the invention being used to deliver a blood pumping device to the left ventricle of the heart via a femoral artery route (FIG. 23), the retrieval catheter advanced via the subclavian vein into the left ventricle to “snag” the proximal end of the power lead (FIG. 24), and the device in the left ventricle coupled to the power unit/controller which is implanted sub dermally in the chest of the subject via the power lead which extends through the subclavian vein, superior vena cava, right atrium, interatrial septum, left atrium and into the left ventricle (FIG. 25). The system of the invention may be configured for the delivery device for the blood pumping device to pull the proximal end of the power lead into the right atrium when the delivery device is being withdrawn. The retrieval catheter may then be advanced into the right atrium and snag the proximal end of the power lead and retrieve it to the subclavian vein where it is coupled with the implanted power unit/controller.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCE NUMERALS

Blood pumping device 1
External tubular housing 2
Blood inlet 3
Blood outlet 4
Rotor 5
Internal lumen 6
Spaced apart bearings 7
Stator 8
PCB (electronic circuitry) 9
Housing proximal part 10
Housing distal part 11
Housing Dividing wall 12
Central annulus 13
Central lumen 15
Impeller device 20
Tubular hub 21
Elongated swept vanes 22
Left atrium 30
Left ventricle 31
Aorta 32
Aortic cusps 33
Mitral valve 34
Right atrium 35
Right ventricle 36
Left ventricular septum 37
Left ventricular apex 38
First anchoring arm 39A
Second anchoring arm 39B
Third anchoring arm 39C
First system of the invention 40
Controller device 41
Power module/unit 42
Controller/processing device 43
Control lead 44
Sensing lead 45
ECG sensor 46
Second system of the invention 50
Sensor 51
Sensor lead 55
Third system of the invention 60
Sensor 61
Sensor 70
Microprocessor/control unit 81
electronic circuitry 83
memory 84.
Blood pumping device 90
Cylindrical proximal part 91
Motor 92
Cylindrical distal part 93
Central part 94
Struts 95
Blood inlet apertures 96
Power/control lead 97
Anchoring barb 98
Blood pumping device 100
Fluidic extension conduit 101
Open distal end 102
Open proximal end 103
Blood pumping device 110
Chamber 111
Loop 112
Retrieval catheter 113
Distal hook 114

1. A system (40, 50, 60) to treat heart failure with preserved ejection fraction (HFpEF), the system comprising:
a blood pumping device (1, 90, 100, 110) configured for implantation in a left ventricle (31) of a heart of a subject, in which the blood pumping device is configured to draw blood from a left atrium (30) into the left ventricle (31) of the heart through a mitral valve (34) upon activation;
an anchoring assembly (39A, 39B, 39C) for anchoring the blood pumping device to a wall of the left ventricle; and
a controller (41, 43) configured to modify the output parameters of the blood pumping device so as to activate and deactivate the blood pumping device in a pattern synergistic with a cardiac cycle of the subject comprising activation during ventricular diastole and deactivation during ventricular systole.

2. A system according to claim 1, further comprising at least one sensor (46, 51, 61, 70) in communication with the controller to detect one or more parameters associated with the heart, wherein the controller is configured to modify the output parameters of the blood pumping device (1) based on the one or more detected parameters received from the at least one sensor.

3. A system according to claim 2, in which the at least one sensor is configured to detect one or more parameters selected from heart rate, closure of the aortic valve, opening of the aortic valve, closure of the mitral valve, and opening of the mitral valve and the controller is configured to adjust the frequency of activation and deactivation of the blood pumping device based on the one or more parameters sensed by the sensor.

4. A system according to claim 4, in which controller (41, 43) is configured to adjust the frequency of activation and deactivation of the blood pumping device by modulating the frequency of the voltage supply to the blood pumping device.

5. A system according to any of claims 2 to 4, in which the at least one sensor (46, 51, 61, 70) is configured to detect a blood pressure parameter of the subject's heart and the controller is configured to adjust the pump flow rate of the blood pumping device to correspond to the blood pressure parameter sensed by the sensor.

6. A system according to claim 5, in which the sensor is an atrial pressure sensor.

7. A system according to claim 5 or 6, in in which controller (41, 43) is configured to adjust the pump flow rate of the blood pumping device by modulating the amplitude of the voltage supply to the blood pumping device.

8. A system according to any preceding claim, in which the blood pumping device (100) comprises a fluidic extension conduit (101) configured to provide fluidic communication from inside the left atrium (30) to the blood inlet (3) of the housing (2).

9. A system according to claim 8, in which the fluidic extension conduit (101) is detachably coupled to the housing (2).

10. A system according to any preceding claim, in which the fluidic extension conduit (101) is flexible.

11. A system according to any preceding claim, in which the blood pumping device (90, 100) comprises:
a first cylindrical part (91) comprising the motor (92);
a second cylindrical part (93) comprising the impeller (20);
a central part (94) connecting the first cylindrical part and second cylindrical part comprising the fluid inlet (3); and
a fluid outlet (4) disposed in the first cylindrical part or second cylindrical part, wherein the rotor (5) extends from the motor to the impeller through the central part.

12. A system according to claim 11, in which the fluid outlet (4) is disposed at a free end of the second cylindrical part (93).

13. A system according to claim 11 or 12, in which the central part (94) of the blood pumping device comprises a plurality of struts (95) connecting the first cylindrical part (91) and second cylindrical part (93), wherein the blood inlet (3) comprises a plurality of apertures (96) defined by the plurality of struts (95).

14. A system according to any of claims 2 to 13, comprising a heart pacing sensor such as an ECG and an atrial pressure sensor.

15. A system according to any of claims 2 to 13, comprising a heart pacing sensor such as an ECG and a ventricular pressure sensor.

16. A system according to any preceding claim, in which the controller (41. 43) is implantable

17. A system according to any preceding claim, including a power unit (42) associated with the blood pumping device, in which the power unit is implantable.

18. A system according to claims 16 and 17, in which the controller (41, 43) and power unit (42) are contained within a single implantable unit.

19. A system according to claim 17 or 18, in which the power unit (42) is configured for contactless induction charging.

20. A system according to claims 16 and 17, including a power lead (97) configured to operatively connected the power unit (42) and/or controller (41, 43) with the blood pumping device.

21. A system according to claim 20, in which the blood pumping device comprises a chamber (111) and the power lead is disposed in the chamber in a spooled or wound configuration.

22. A system according to claim 21, comprising a retrieval catheter (113) configured to capture a proximal end of the power lead (97) and pull the power lead from the chamber (111).

23. A system according to any preceding claim, comprising an access sheath having a lumen configured for percutaneous delivery of the blood pumping device to the left ventricle.

24. A system according to any preceding claim, comprising a delivery shaft for the blood pumping device to advance the blood pumping device through the lumen of the access sheath.

25. A system according to claim 24, in which the delivery shaft and blood pumping device are configured for detachable coupling together.

26. A system according to any preceding claim, in which the anchoring assembly comprises a plurality of anchoring arms (38A, 39B, 39C) configured for adjustment from a stowed position suitable for percutaneous delivery to a deployed position in which the plurality of anchoring arms oppose the ventricular wall.

27. A system according to claim 26, in which the anchoring arms are configured for self-deployment.

28. A system according to any preceding claim, in which the anchoring assembly is configured to couple to the blood pumping device in-vivo.

29. A system according to claim 29, in which the anchoring assembly comprises an anchoring hub configured for coupling to the blood pumping device and a plurality of anchoring arms extending from the hub.

30. A system according to claim 11, in which the impeller (20) comprises an axial hub (21) and at least two vanes (22) mounted to the hub, in which each vane has an elongated swept profile.

31. A system according to claim 30, in which each vane (22) comprises a hub to tip ratio (v) of 0.20 to 0.30.

32. A system according to claim 30 or 31, in which the impeller (20) comprises two vanes (22) disposed on opposed sides of the hub.

33. A system according to any of claims 30 to 32, in which each vane (22) has an axial length of 10 to 15 mm and a radial width of 3 to 7 mm.

34. A system according to any of claims 30 to 33, in which each vane (22) extends around the hub along a sweep angle of 80° to 120°.
